(19) United States
(12) Patent Application Publication
(10) Pub. No.: US 2012/0065695 A1
(43) Pub. Date: Mar. 15, 2012

(54) MEDICAL WIRE

(75) Inventor: Ken Ishii, Tokyo (JP)

(73) Assignee: KEIO UNIVERSITY, Tokyo (JP)

(21) Appl. No.: 13/264,887

(22) PCT Filed: Apr. 27, 2010
(86) PCT No.: PCT/JP2010/003025

§ 371 Date: Nov. 28, 2011

Related U.S. Application Data
(60) Provisional application No. 61/213,001, filed on Apr. 27, 2009.

(51) Int. Cl. A61B 17/58

(52) U.S. Cl. 606/86 R

(57) ABSTRACT

The present invention provides a medical wire capable of significantly reducing the risk of its frontal end to puncture an anterior wall of a bone and to move out of the bone, thereby greatly improving safety of a surgery. The medical wire includes an end composed of a material that deforms to increase resistance during advancement in the bone and substantially regains initial shape during retreat from the bone.

### TECHNICAL FIELD

The present invention relates to medical wires, and in particular medical wires whose one end is to be inserted into a bone, and are useful as a guide wire for preventing its movement inside/outside of a bone during a spinal surgery, as a wire for fixing bones in treatment of bone fracture, as well as a guide wire to be used in an placement of a fixation implant.

### BACKGROUND ART

In the medical field of orthopedic surgery, steel wires which are called Kirshner wires (K-wires) have been widely used for osteosynthesis, arthroplasty, and so on.

More recently, in the field of spinal surgeries, steel guide wires have become popularly used in minimally invasive surgeries (MIS) to implant biomaterial, such as posterior spinal fusion, and in particular, transforaminal lumbar interbody fusion (TLIF), also known as posterior lumbar interbody fusion (PLIF), where an endoscope is used through micro-incision to fix an unstable backbone which has been dislocated and has compressed nerves.

The TLIF can be applied to diseases in which the backbone is dislocated or the inter-vertebral disk between vertebrae is injured to damage the nerves running within the backbone (spinal canal), causing lumbago, leg pain and/or numbness. Specific diseases include lumbar disc disease, lumbar disc herniation, lumbar degenerative spondylolisthesis, lumbar spinal canal stenosis, lumbar degenerative scoliosis, lumbar isthmic spondylolisthesis, scoliosis, traumatic injuries such as bone fractures, metastatic tumor in vertebra, and the like.

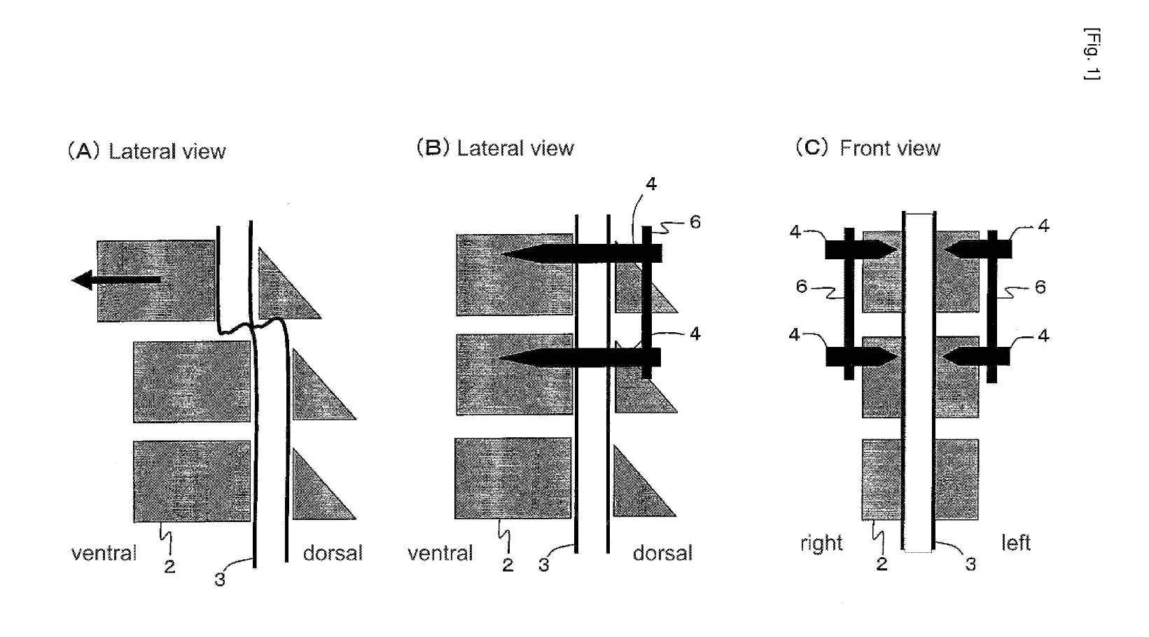

In an exemplary lumbar spinal canal stenosis due to a spondylolisthesis, as illustrated in FIG. 1(A) showing a lateral view of a backbone 2, the backbone 2 is dislocated to cause severe nerve compression in a patient whose walking distance is limited to approximately 20 m and is suffering from incontinence. A surgery is performed to the patient, by which screws 4 are implanted from the back and fixed by rods 6 as illustrated in FIG. 1(B) showing a lateral view of the backbone 2 and (C) showing a front view of the backbone 2 to correct the location of the backbone 2 and widen the passageway of the nerve (spinal canal) 3, thereby releasing the nerve compression and enabling unlimited walking distance and possible improvement of the incontinence.

In the surgery, the back of the patient in prone position is incised by about 3 cm, and after visibility is secured by using a pipe away from muscles, an endoscope is inserted, the nerve compression is released, and an implant called the cage is placed between the vertebral bodies. Then, as illustrated in FIG. 1(B) and (C), four of the screws 4, each two of which are inserted into a vertebra in fixing two vertebra, are implanted to the backbone 2 to correct its dislocation, and finally the screws 4 are connected by the metal rods 6.

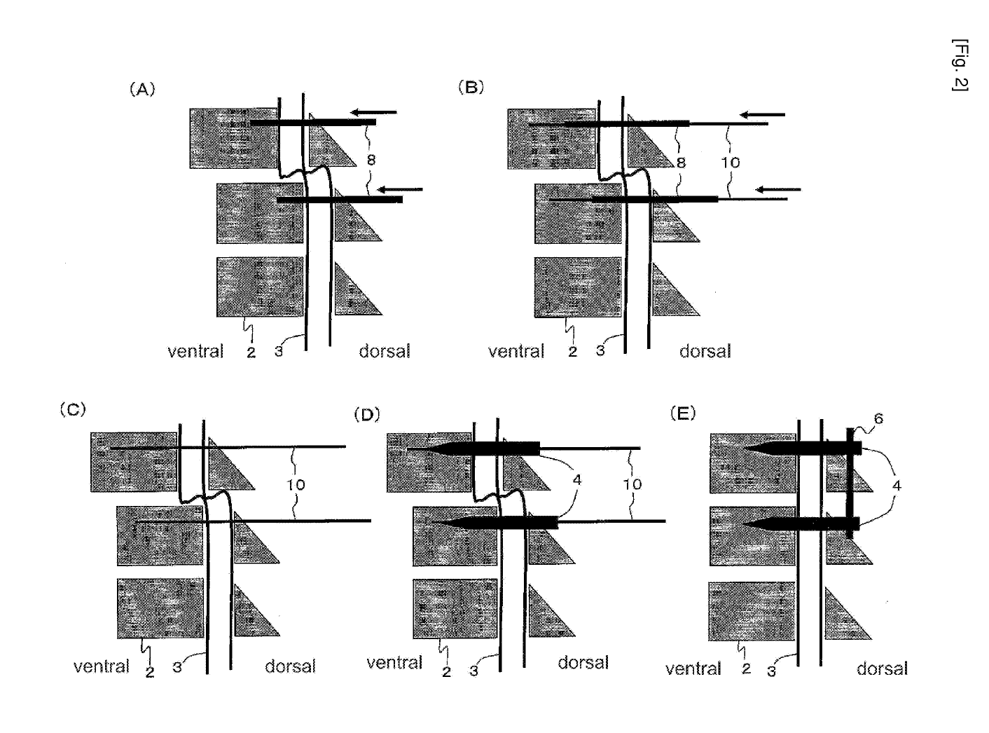

To insert the screw 4, a relatively thick double-needle 8 with an outer diameter of about 4 to 5 mm, which is called a starting needle, a target needle, or pack needle, is implanted into the backbone 2 under X-ray fluoroscope at first as illustrated in FIG. 2(A). The inner needle is removed and substituted by a thin guide wire 10 with a diameter of about 1.0 to 2.0 mm as in FIG. 2(B), and then the outer sheath of the starting needle is also removed as in FIG. 2(C). Using the remaining guide wire 10 as a guide, thread-cutting performed with a tap etc, and as shown in FIG. 2(D), a hollow screw (pedicle screw) 4 is implanted. The guide wire 10 is finally removed through the inside of the screw 4.

Afterwards, corrective force is applied to the inserted screw 4 as in FIG. 2(E), to move the displaced backbone 2 to a normal position, thereby widening the passageway of the nerve 3.

More than thirty companies are offering unique biological fixation devices or systems, all of which utilize a guide wire, characterizing this particular technique of the surgery(Japanese Patent Application Publication Nos.2007-513739 and 2007-506514).

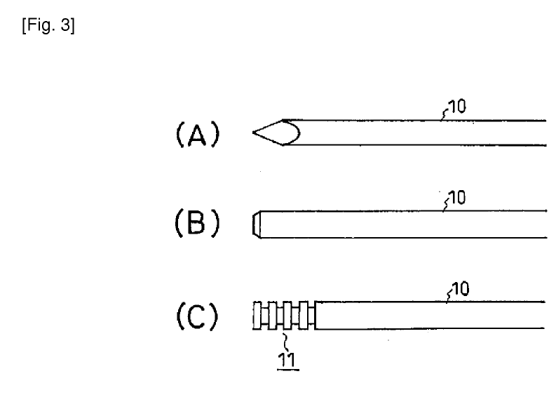

As for the guide wire 10, generally used are the wire as shown in FIG. 3(A) whose one end is made into a sharp conical shape to enable easy insertion into a bone (hereinafter, the “sharp-end wire”), as well as the wire with as in FIG. 3(B), in which one end of a cylindrical wire is blunted by chamfering (hereinafter “blunt-end wire”).

In addition to MIS-TLIF, guide wires are also used in plastic operations for vertebral body. The plastic operation for vertebral body is performed to stabilize the vertebra fractured due to a compression fracture etc. and to relieve pains, in which a surgeon injects bone cement or artificial bone into the fractured site with an aid of X-ray fluoroscope.

In a conventional plastic operation for vertebral body called vertebroplasty, the bone cement is injected directly into the collapsed vertebra (Orthop Clin North Am. 2009 Oct;40(4):465-71, viii.). However, the cement is often leaked out of the vertebra, causing various complications as reported. Further, this method can hardly correct the deformation of the vertebral body, and the effect of the operation is limited.

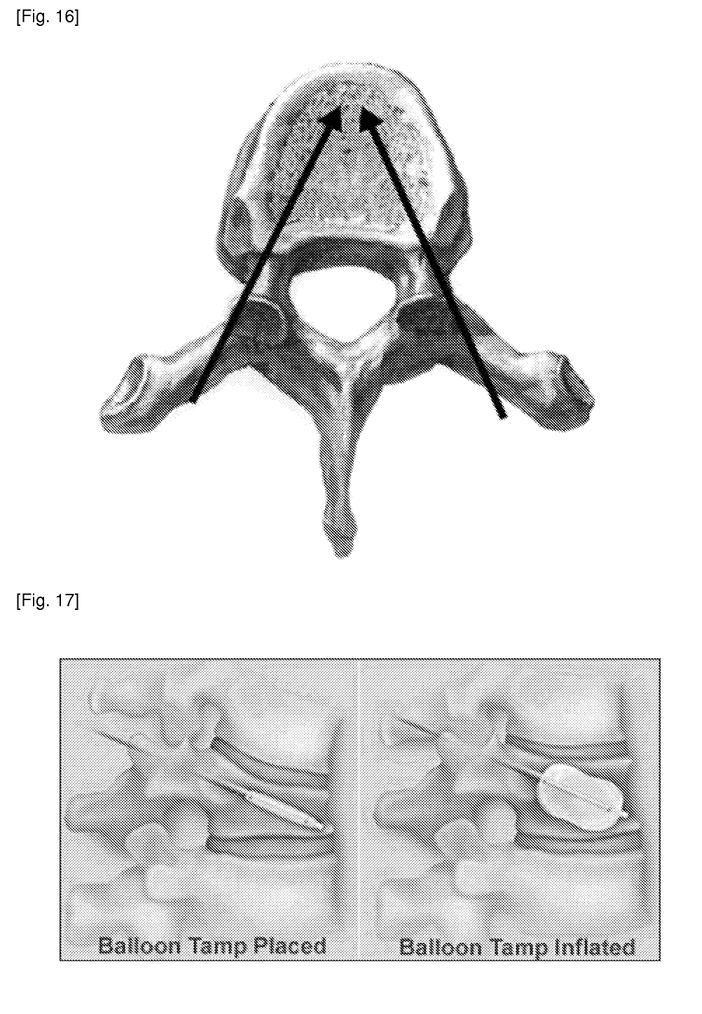

Then, another type of plastic operation for vertebral body called kyphoplasty has been developed (Orthop Clin North Am. 2009 Oct;40(4):465-71, viii.). In this method, a needle is transdermally inserted into the posterior vertebral body via a pedicle under the guide of X-ray fluoroscope like MIS-TLIF (FIG. 16). Then, a guide wire is inserted into the vertebral body through the needle, the needle is removed, and a cannula is inserted via the guide wire. After removing the guide wire, a bone tamp having a balloon at its one end, such as KyphX Xpander Inflatable Bone Tamp (Medtronic Inc.), is inserted into the vertebral body through the cannula (FIG. 17). The balloon is inflated to secure a height of the vertebral body, then the bone tamp is removed, and a resulting open space within the vertebral body is filled with bone cement such as polymethyl methacrylate cement (Medtronic Inc.). This method provides greater improvement after the operation and reduces the leakage of the cement.

Further, a guide wire has been recently used in vertebral augmentation (Euro Spine J., published online on Mar. 01, 2010, Springer).

### SUMMARY OF INVENTION

### Technical Problem

The abovementioned methods of surgery utilizing the guide wire do not require a large-scale incision in the back and therefore are minimally invasive in essence. However, they could still accompany various complications during the implantation of screws. The representative complications are (1) severe intestinal injuries and vascular injuries caused by penetration of the guide wire 10 from the backbone 2 to a front side as illustrated in FIG. 4(A) and (B) (in a direction to the left in FIG. 4(A) and to the bottom of FIG. 4(B)); (2) nerve injuries caused by penetration of the guide wire 10 or screw 4 into the spinal canal as illustrated in FIG. 4(B); and (3) insufficient fixation etc. due to loosening of the screw. In particular, the most serious complication is damage to intestine or vessel caused by anterior penetration of the guide wire. The vertebra are surrounded in particular by visceral tissues such as great vessels and gastrointestinal tracts as well as nerve tissues as illustrated in FIG. 4(B), and thus an unintentional movement of the guide wire 10 inserted into the backbone 2, such as penetration out of the vertebra through a puncture in the anterior wall, would let the guide wire penetrate through an anterior bone cortex to reach the posterior peritoneum or the peritoneal cavity, and cause damages to these important tissues. In particular, an injury of the great vessel would be fatal, and would make life-saving difficult. An intestinal injury also often leads to a serious condition.

Since the use of the guide wire is a prerequisite in the currently used MIS-TLIF (PLIF) systems, such critical complications are always conceivable.

Although a guide wire having a circumferential grooves 11 on its one end as shown in FIG. 3(C), as well as a guide wire having a slightly thinner end are available, they have been developed essentially to prevent a sudden slipping-out of the wire during the surgery, and do not provide preventive effect against the frontal movement out of the backbone. Rather, they have smaller resistance to the frontal movement due to the smaller diameter of the end portion, and thus the blunt-end wires shown in FIG. 3(B) are used more commonly in actual clinical applications from the viewpoint of safety.

The present invention has been made in order to solve the abovementioned problems, and is intended to provide a medical wire capable of limiting its movement within a bone when its one end is inserted into the bone, as well as preventing its movement out of the bone or perforation of the bone.

### SOLUTION TO PROBLEM

In an embodiment, a medical wire having an end to be inserted into a bone, the end being constituted so that the end deforms to increase resistance during advancement in the bone and substantially regains an initial shape of the end portion during retreat from the bone.

In an embodiment, the end may include a constituent wire thinner than the main body of the wire. The wire may have a multiple of the constituent wires. The constituent wires may be braided, stranded or bundled. The constituent wire may be coiled. The medical wire may be a pipy hollow wire and the constituent wire may be inserted in the end portion of the hollow wire.

In another embodiment, the end may be composed of a flexible material. The flexible material may be a shape memory metal such as Nitinol, rod-like rubber or plastic.

In an embodiment, the medical wire may be used in a spinal surgery. In another embodiment, the medical wire may be used in an implantation of an internal/external fixation device for a treatment of bone fracture. In further embodiment, the medical wire may be used in a plastic operation for vertebral body such as vertebroplasty, kyphoplasty and vertebral augmentation.

The present invention also provides a use of a medical wire including the steps of inserting the medical wire into a bone, allowing an end of the medical wire to deform to increase resistance within the bone, allowing the end of the medical wire to substantially regain initial shape of the end, and removing the medical wire from the bone.

### CROSS REFERENCE TO RELATED APPLICATIONS

The present application claims the benefit of priority to the U.S. Provisional application Ser. No. 61/213,001 filed on Apr. 27, 2009, the disclosure of which is herein incorporated by reference.

### ADVANTAGEOUS EFFECTS OF INVENTION

The present invention can provide a medical wire having an end which bends moderately to produce resistance when an advancing force is applied to the wire, significantly reducing the risk of the medical wire to move within a bone and/or to move out of the bone to puncture an anterior wall of the bone, thereby greatly improving safety of a surgery. Even if the wire sharply bends at a flexible part during the advancing movement, it can regain a shape similar to the initial shape during retreat from the bone, thereby enabling smooth removal of the wire.

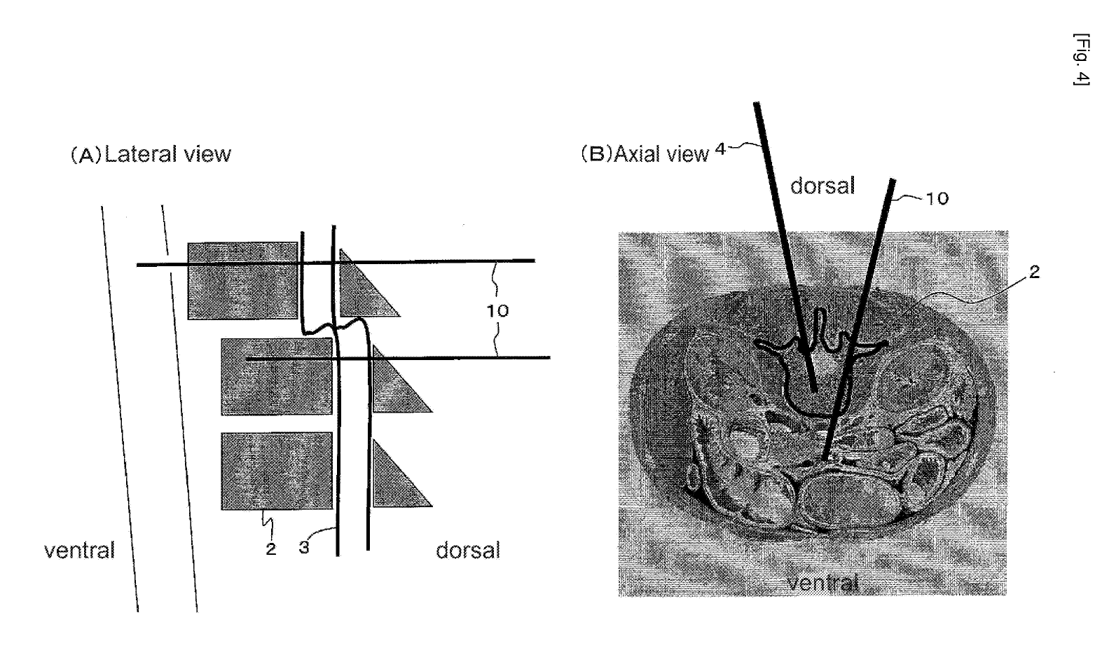

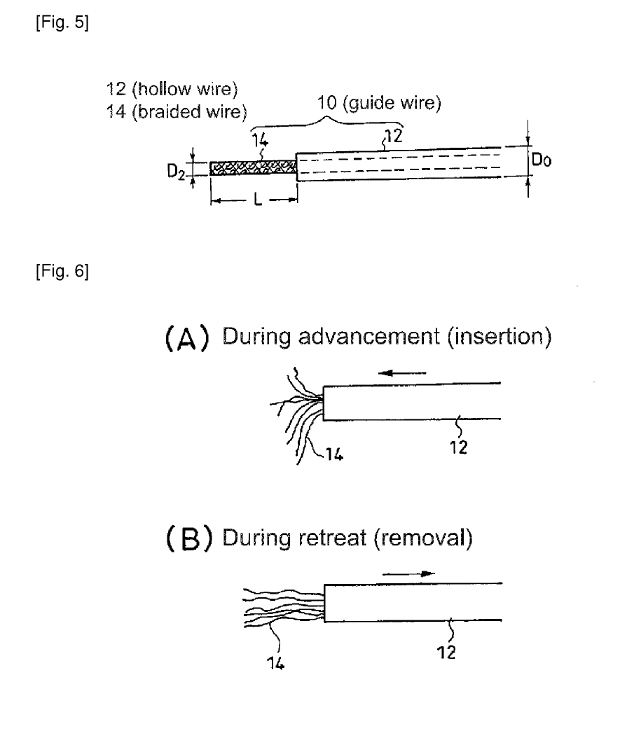

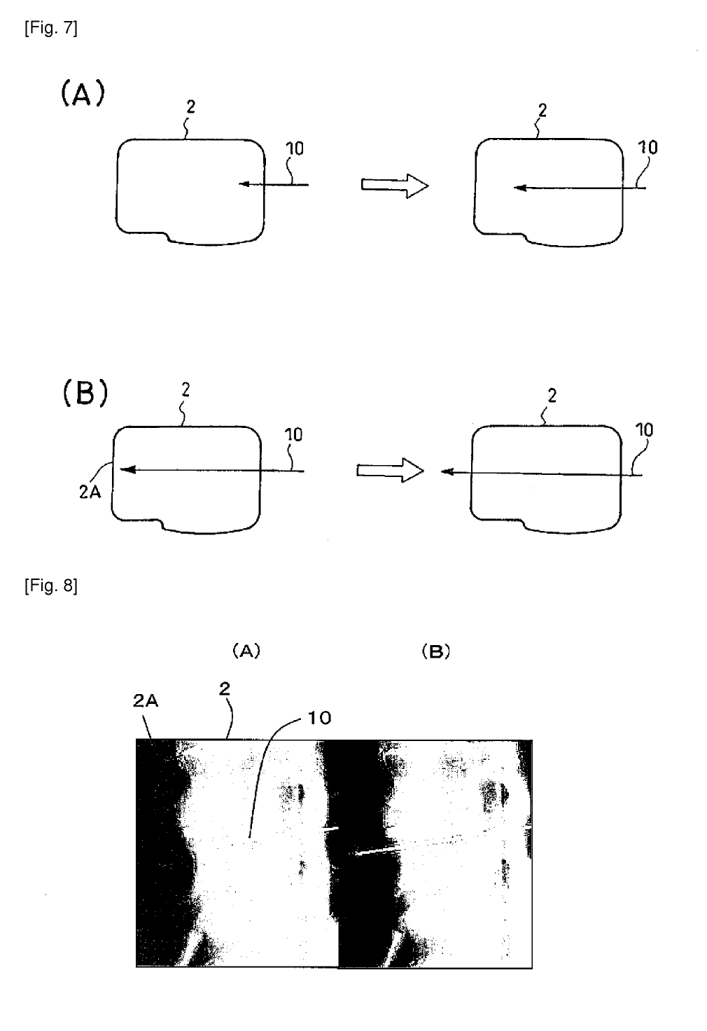

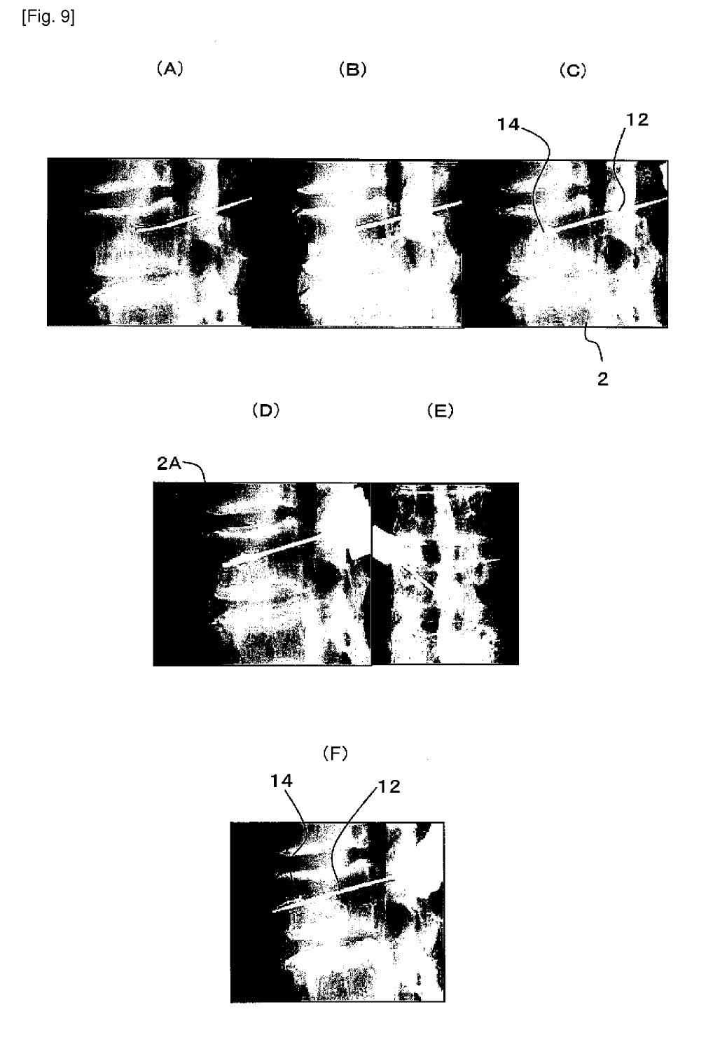

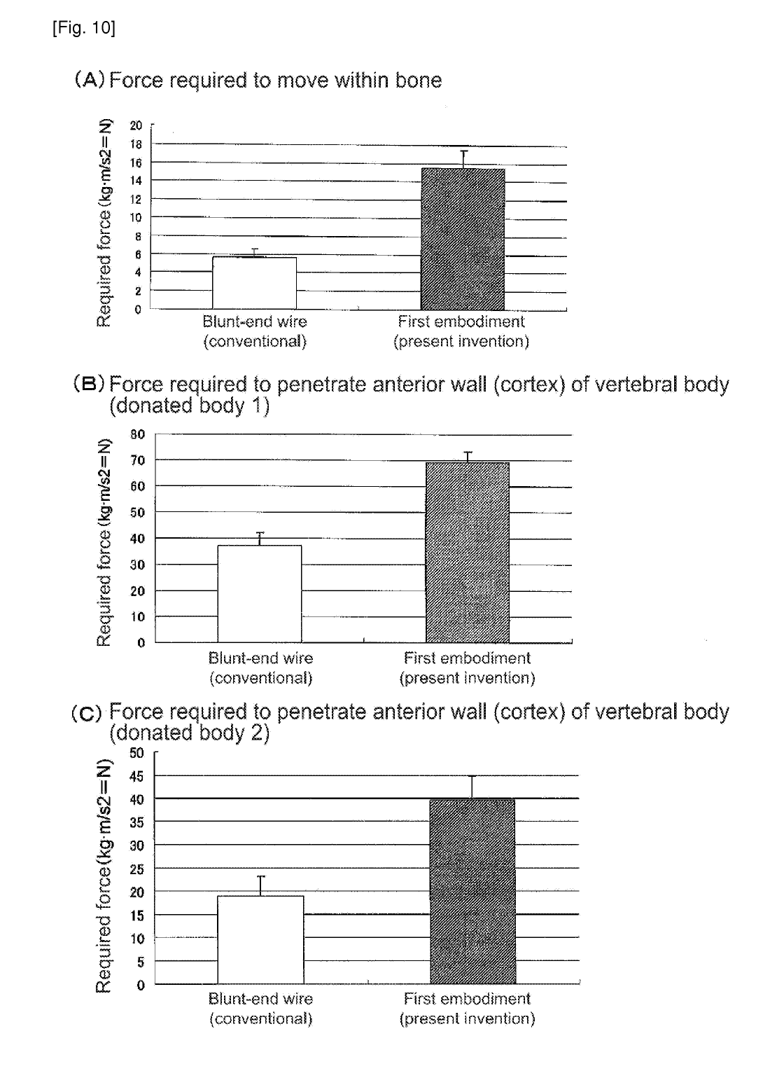

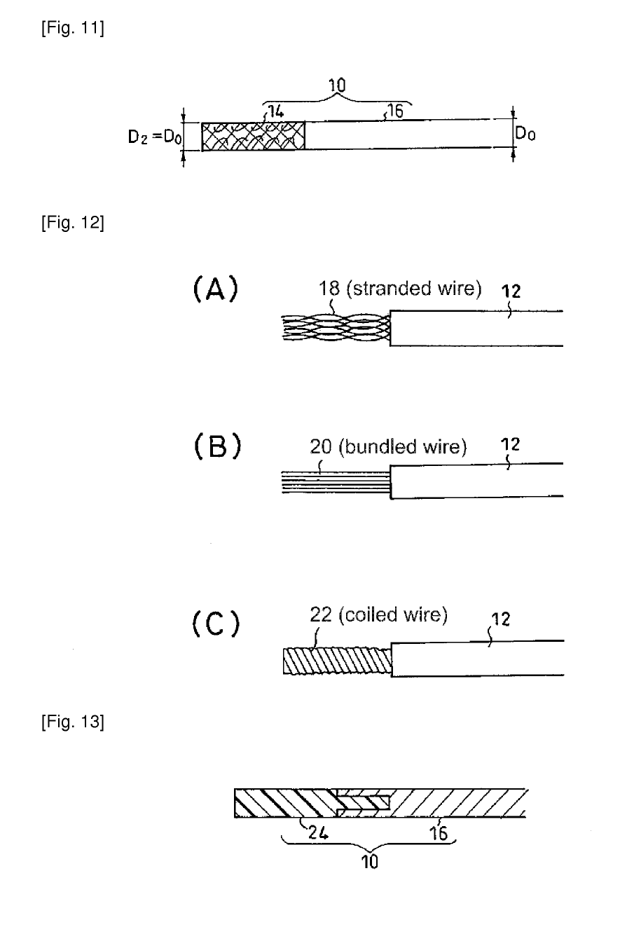

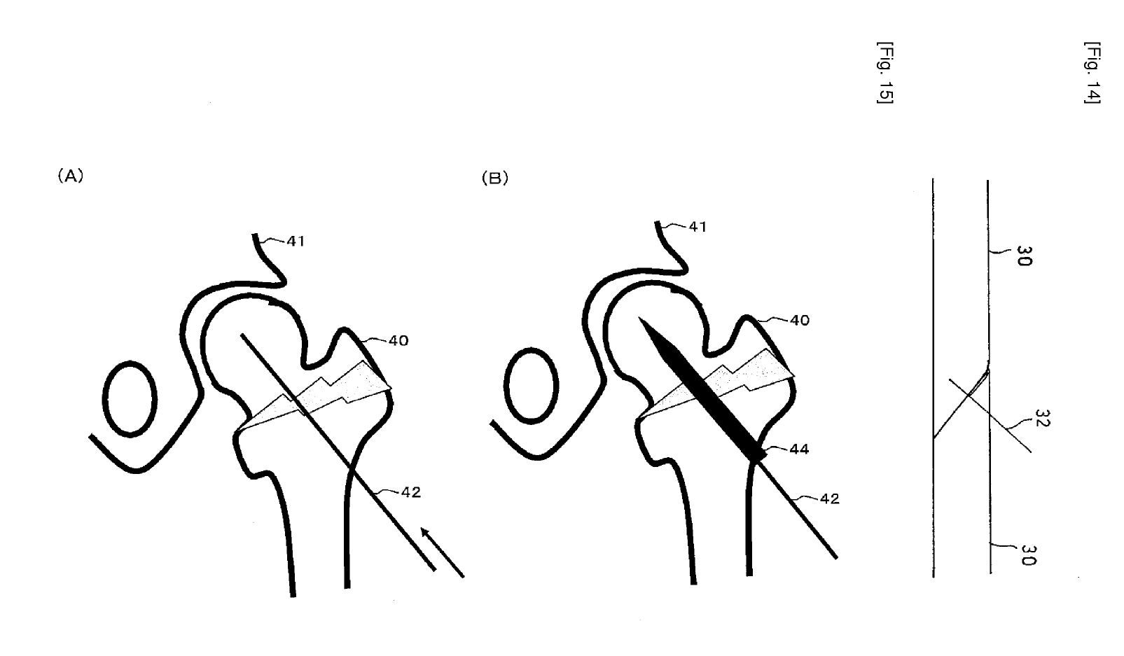

### BRIEF DESCRIPTION OF DRAWINGS

[FIG. 1]FIG. 1 shows an exemplary spinal surgery, i.e. vertebral fixation.

[FIG. 2]FIG. 2 shows the procedure to insert screws into vertebra.

[FIG. 3]FIG. 3 shows shapes of the end of the conventional guide wire.

[FIG. 4]FIG. 4 shows scheme to explain severe complications when an end of the guide wire penetrates during the insertion of pedicle screw.

[FIG. 5]FIG. 5 shows a configuration of a first embodiment of the present invention.

[FIG. 6]FIG. 6 shows actions of the first embodiment of the present invention.

[FIG. 7]FIG. 7 shows a diagram of an experiment using a donated body.

[FIG. 8]FIG. 8 shows a movement of a conventional blunt-end wire in the experiment using a donated body (X-ray fluoroscopy images).

[FIG. 9]FIG. 9 shows the movements of the guide wire in a donated body in the first embodiment of the present invention (X-ray fluoroscopy images).

[FIG. 10]FIG. 10 shows the comparison of results of the experiments.

[FIG. 11]FIG. 11 shows the ends of the guide wires in the second embodiment of the present invention.

[FIG. 12]FIG. 12 shows the variations of the wire other than the braided wire.

[FIG. 13]FIG. 13 shows a cross-sectional view of an end of the guide wire in a third embodiment of the present invention.

[FIG. 14]FIG. 14 shows the medical wire according to the present invention being used for fixation of a fractured bone.

[FIG. 15]FIG. 15 shows the medical wire according to the present invention being used for treatment of a fractured bone in a hip joint.

[FIG. 16]FIG. 16 shows an embodiment in which a needle is inserted into a vertebral body in kyphoplasty.

[FIG. 17]FIG. 17 shows an embodiment in which a balloon is introduced into a vertebral body (left panel) and inflated (right panel) in kyphoplasty.

### DESCRIPTION OF EMBODIMENTS

Hereinafter embodiments of the present invention are described in detail with reference to drawings.

It should be noted that the object, characteristics, advantages and ideas of the present invention will be apparent to those skilled in the art from the descriptions in the present specification, and the present invention can be easily reproduced by a person skilled in the art based on the descriptions in the present invention. The embodiments and specific examples of the invention described herein are to be taken as preferred embodiments of the present invention, and are presented only for illustrative and/or explanatory purposes but not to limit the present invention. It is further apparent to those skilled in the art that various changes and modifications may be made based on the descriptions in the present specification within the intent and scope of the present invention disclosed herein.

### CONFIGURATION OF MEDICAL WIRE

A medical wire according to the present invention is not limited as long as it has an end which is constituted so that the end can deform to increase resistance during advancement in a tissue and substantially regains an initial shape during retreat from the bone. Embodiments of the configurations of the medical wire are explained below.

In a first embodiment of the present invention, a medical wire 10 consists of a pipy hollow wire 12 of stainless steel having an end into which a braided wire 14 formed of thin braided constituent wires of stainless steel is squeezed, as illustrated in FIG. 5.

The outer diameter Dₒ of the hollow wire 12 may be similar to the outer diameter of a conventional guide wire, for example in the range of 1.0 to 5.0 mm, preferably, 1.0 to 3.0 mm, or more preferably 1.0 to 2.0 mm.

The outer diameter D₂ of the braided wire 14 may be about 1 mm for example, and the length L of the braided end protruding out of the hollow wire 12 may be in the range of 5 to 15 mm for example and preferably about 10 mm. The length L should be adjusted appropriately, because a too long L would make the guide wire so difficult to operate, whereas a too short L would reduce the resistance so much to prevent an unintended slipping.

The first embodiment of the medical wire can be quite easily manufactured because the braided wire 14 may be simply squeezed into the end of the hollow wire 12. The hollow wire 12 may be replaced by a solid wire having a hole in the end. The material of the medical wire is not limited to stainless steel, and may be another kind of metal such as copper or Nitinol.

When the medical wire of this embodiment is used as a guide wire, the braided end becomes moderately unwoven as it is inserted and advances in a tissue such as bone, as illustrated in FIG. 6(A), increasing resistance against the advancement of the guide wire and applying the brake. Even if its braided end becomes unwoven and bends during the advancement, it can regain a shape similar to the initial shape during retreat of the end at the removal of the medical wire, as illustrated in FIG. 6(B), enabling smooth removal of the medical wire after the screws are implanted. This mechanism may be realized by a configuration in which the braided end is loosened by winding the wire clockwise and tightened by winding it anticlockwise.

In addition to the first embodiment of the medical wire in which the braided wire 14 is inserted in the end of the hollow wire 12, a braided wire 14 having the same outer diameter as a solid wire 16 (i.e. D₂=D₀) may be connected to an end of the solid wire 16 by welding etc. in a second embodiment as illustrated in FIG. 11.

What may also be used in place of the braided wire 14 formed of the woven constituent wires in these embodiments are: a stranded wire 18 in which constituent wires are spirally twined as in FIG. 12(A); a bundled wire 20 in which constituent wires are simply bundled as in FIG. 12(B); and a coiled wire 22 in which one or a few constituent wires are coiled as in FIG. 12(C).

In the third embodiment as illustrated in FIG. 13, a rod-like flexible material 24, a shape memory metal such as Nitinol, rubber or plastic having elasticity and being deformable may be inserted into an end of a solid (or hollow) wire 16 composed of metal and fixed by glue etc.

While the medical wire in each of the preceding embodiments of the present invention can be used as a guide wire for a spinal surgery, a medical wire 32 having a larger diameter of about 1 to 5 mm, or preferably 3 to 5 mm may also be used as a internal fixation device for fixing a fractured bone such as a long bone 30 as illustrated in FIG. 14.

Further, the medical wire of the present invention may also be used as a guide wire 42 for an insertion of an internal/external fixation device such as a screw implant 44 in treatment of bone fracture of femoral neck 40 in a hip joint as illustrated in FIG. 15(A) and (B). FIG. 15(A) shows the guide wire 42 being inserted to penetrate the fractured bone, and FIG. 15(B) shows the screw implant 44 being inserted along the guide wire 42. In this example, the present invention is particularly useful because it is prevented from slipping of an end into the pelvic cavity 41, which could cause damages to organs or vessels in the pelvic cavity and lead to massive bleeding.

While in each of the preceding embodiments the medical wire is used as a guide wire in a surgery, it may be used in applications other than surgeries, such as other kinds of treatments and diagnosis.

<Use of Medical Wire>

A guide wire in the embodiments of the present invention may be used to insert a hollow device such as a cannula or a screw into a bone. Specifically, a first hollow device such as a needle is inserted into the bone. Then, the guide wire is inserted into the first hollow device and pushed into the bone. By this advancement of the guide wire, the frontal end of the wire may be deformed to increase resistance against the advancement in the bone. For example, a braided wire, a stranded wire, a bundled wire or a coil at the end of the medical wire may become unwoven to deform. When the guide wire is pushed into to a predetermined position, the first hollow device is removed. Then, a second hollow device such as a cannula or a screw is inserted into the bone with the guidance of the guide wire. Once the second hollow device is inserted to a predetermined position, the guide wire is pulled back and the end of the guide wire may regain a shape similar to its initial shape by retreating movement of the guide wire. The guide wire is pulled back further and is removed from the bone. The type, position etc. of the bone is not particularly limited, but the bone is preferably a vertebral body of a vertebra.

More specifically, the medical wire according to the present invention may be used in posterior spinal fusion, in particular, posterior lumber interbody fusion (MIS-TLIF or MIS-PLIF). The medical wire according to the present invention may be applied to any disease that involves a dislocation of backbone, such as lumbar disc disease, lumbar disc herniation, lumbar degenerative spondylolisthesis, lumbar spinal canal stenosis, lumbar degenerative scoliosis, lumbar isthmic spondylolisthesis, scoliosis, traumatic injuries such as bone fracture, metastatic tumor in vertebra and the like.

In a method of inserting screws to correct dislocation of adjacent vertebral bodies, a hollow needle is inserted into each of two or more adjacent vertebral bodies. Then guide wires according to the present invention are inserted into the needles and pushed into the vertebral bodies. During advancement of the guide wires, the frontal ends of the wires are deformed to increase resistance in the vertebral bodies. When the guide wires are each pushed into to a predetermined position, the needles are removed. Then hollow screws are inserted into the vertebral bodies with the guidance of the guide wires. Once each of the screws are installed at a predetermined position, the guide wires are pulled back and their ends regain a shape similar to its initial shape during retreating movement of the guide wire. The guide wires are pulled back further and removed from the vertebral body. Then, a force for correcting the bone is applied to the inserted screw to restore the dislocated vertebral bodies.

In this embodiment, the hollow needle such as back needle preferably has an inner diameter in the range of 1 to 3 mm and an outer diameter in the range of 2 to 5 mm. Alternatively, a relatively thick double-needle with an outer diameter of about 4 to 5 mm, called a starting needle, a target needle, or pack needle may be inserted, from which an inner needle may be then removed, and a guide wire may be inserted in place of the inner needle. Preferably the screw has a diameter in the range of 3 to 7 mm.

The guide wire according to the present invention may be used in other application such as plastic operation for vertebral body including vertebroplasty, kyphoplasty and vertebral augumentation. It may be applied to any disease that requires plastic operation of vertebral body, such as bony metastasis of tumor into a vertebral body, compression fracture accompanying osteoporosis, blow-out fracture and the like.

In the method of the plastic operation, a hollow needle is introduced into a posterior vertebral body. Then, a guide wire is inserted into the needle and pushed into the vertebral body. During advancement of the guide wire, its frontal end is deformed to increase resistance in the vertebral body. When the guide wire is pushed into to a predetermined position, the needle is removed. Then a hollow cannula is inserted into the vertebral body with the guidance of the guide wire. Once the cannula is inserted to a predetermined position, the guide wire is pulled back and its end regains a shape similar to its initial shape during retreating movement of the guidewire. The guide wire is further pulled back and removed from the vertebral body.

In the case of vertebroplasty, bone cement may be then injected through the cannula.

In the case of kyphoplasty, a bone tamp having a balloon at its one end may be inserted into the vertebral body through the cannula, the balloon is inflated to secure a height of the vertebral body, the balloon or the bone tamp is removed, and a resulting open space within the vertebral body is filled with bone cement. In the case of vertebral augmentation, metal around the balloon is inflated together with the balloon to secure a height of the vertebral body, the balloon or the bone tamp is removed, and a resulting open space within the vertebral body is filled with bone cement.

In this embodiment, the hollow needle preferably has an inner diameter in the range of 3 to 5 mm and an outer diameter in the range of 3 to 8 mm. The cannula preferably has an inner diameter in the range of 3 to 5 mm, and an outer diameter in the range of 3 to 8 mm. The bone cement may be for example hydroxyapatite or polymethyl methacrylate.

### EXAMPLES

Safety of the medical wire in the first embodiment of the present invention was proved by an experiment as follows. Since it is impossible to demonstrate the usefulness of the wire by penetrating a bone in an actual surgery, three pieces of fresh bones from donated bodies were used to conduct the experiment. Under an X-ray fluoroscopy, ten medical wires were inserted to both sides of first to fifth lumbar vertebra in each of the individual bodies. Then the following forces are measured: (1) the force required for an intramedullary movement of the medical wire 10 (the force for advancement by 1 cm in the bone marrow) as shown in FIG. 7(A); and (2) the force required to penetrate the anterior bone cortex 2A of the backbone 2 as shown in FIG. 7(B). Since bone densities were individually variable, a mean value of the penetrating forces was calculated for five vertebral bodies in each of the individuals. A conventional blunt-end wire was inserted from the right pedicle of the vertebral arch and the medical wire according to the first embodiment of the present invention was inserted from the left pedicle of the vertebral arch

Typical movements of a conventional blunt-end wire and a medical wire according to the first embodiment of the present invention are shown in side view of X-ray images in FIG. 8 and FIG. 9, respectively. As shown in FIG. 8(A), the conventional blunt-end wire easily reached an anterior wall 2A of the backbone 2 by a subtle force applied for insertion of the wire, and an additional force caused a penetration of the backbone 2, as well as fast advancement after the penetration, as shown in FIG. 8(B). In contrast, although the inserted medical wire of the first embodiment as shown in FIG. 9(A) did move toward the frontal direction (to the left in the figure) at first by an addition of an advancing force as shown in FIG. 9(B), an frontal end of the wire became moderately unwoven as shown in FIG. 9(C) and further advancement was prevented due to the resistance of the unwoven portion. The medical wire also showed resistance against pull-out due to the unwoven portion toward the direction from which the wire was inserted. The wire then stopped when it reached the anterior wall 2A of the bone 2 as shown in FIG. 9(D). This situation can be viewed from the frontal side as shown in FIG. 9(E) where the frontal end of the medical wire bended and turned toward an internal direction.

An addition of further force finally resulted in a penetration into the anterior wall 2A as shown in FIG. 9(F), but the bent end provided resistance so that a fast unintended protrusion to the front side was prevented even after the penetration. Since the bent portion has elasticity, it does not cause severe damage to the surrounding tissue as much as the conventional blunt-end wire.

As illustrated in FIG. 10(A), the forces required for the movement of the medical wires in the bone were measured using a certain donated body, and found to be 5.68+/−0.82N for the conventional blunt-end wire, versus 15.48+/−1.89N for the medical wire of the first embodiment, indicating a significantly larger resistance of the medical wire of the first embodiment by a factor of about 2.73 (P<0.0001: n=5). In other words, the medical wire according to the present invention is safer because it requires a force 2.73 times more than the conventional blunt-end wire to move in the movement.

Further, as illustrated in FIG. 10(B), the forces required to penetrate the anterior wall (bone cortex) of the backbone were measured in the donated body 1 and found to be 37.07+/−4.81N for the conventional blunt-end wire, versus 69.08+/−4.20N for the medical wire of the first embodiment (P<0.0005: n=5). In another donated body 2, as illustrated in FIG. 10(C), the measured values were 18.67+/−4.30N versus 39.54+/−5.35N (P=0.0228: n=5), indicating a significantly larger resistance of the medical wire of the first embodiment by a factor of about 1.86 in average. In other words, the medical wire of the first embodiment is safer because it requires a force 1.86 times more than the conventional blunt-end wire to be moved out of the bone (bone perforation).

### INDUSTRIAL APPLICABILITY

The medical wire according to the present invention can be used as a guide wire for preventing movement inside/outside of a bone during a spinal surgery, as an internal (or external) fixation device for a treatment of bone fracture, as well as a guide wire to be used in an implantation of a fixation device.

1. A medical wire comprising an end to be inserted into a bone, the end being constituted so that the end deforms to increase resistance during advancement into the bone and substantially regains an initial shape of the end during retreat from the bone.

2. The medical wire according to claim 1, wherein the end comprises a constituent wire thinner than the main body of the wire.

3. The medical wire according to claim 2, wherein the end comprises a multiple of the constituent wires.

4. The medical wire according to claim 3, wherein the constituent wires are braided, stranded or bundled .

5. The medical wire according to claim 2, wherein the constituent wire is coiled.

6. The medical wire according to claim 2, wherein the wire is a pipy hollow wire and the constituent wire is inserted in the end of the hollow wire.

7. The medical wire according to claim 1, wherein the end is composed of a flexible material.

8. The medical wire according to claim 7, wherein the flexible material is a shape memory metal, a rod-like rubber or plastic.

9. The medical wire according to claim 1, wherein the medical wire is used in a spinal surgery.

10. The medical wire according to claim 1, wherein the medical wire is used in an implantation of an internal/external fixation device for a treatment of bone fracture.

11. The medical wire according to claim 1, wherein the medical wire is used in a plastic operation for vertebral body.

12. The medical wire according to claim 11, wherein the plastic operation for vertebral body is vertebroplasty, kyphoplasty or vertebral augumentation.
